(19) United States
(12) Patent Application Publication
(10) Pub. No.: US 2022/0133215 A1
(43) Pub. Date: May 5, 2022

(54) METHOD FOR EVALUATING SKIN LESIONS USING ARTIFICIAL INTELLIGENCE

(71) Applicant: FOTOFINDER SYSTEMS GMBH, Bad Birnbach (DE)

(72) Inventor: Andreas Mayer, Passau (DE)

(21) Appl. No.: 17/434,466

(22) PCT Filed: Oct. 28, 2019
(86) PCT No.: PCT/DE2019/200121

§ 371 Date: Aug. 27, 2021

(30) Foreign Application Priority Data
Feb. 28, 2019 (DE) 10 2019 105 152.5

(51) Int. Cl. A61B 5/00; G16H 30/40; G16H 30/20; G16H 50/20; G06T 7/00

(52) U.S. Cl. CPC A61B 5/444; G16H 30/40; G06N 3/08; G16H 50/20; G06T 7/0012; G16H 30/20

(57) ABSTRACT

The present invention relates to a method for displaying at least one image of a skin lesion and associated information to assist in characterizing the skin lesion, the method comprising the steps of capturing a picture, in particular a close-up picture, of a skin lesion (13) in an area of skin to be examined by means of optical capturing means (2) configured for this purpose, in particular a video dermatoscope, and providing image data based thereon, analyzing the skin lesion by electronically processing the provided image data by means of an artificial neural network configured to identify and/or classify skin lesions, and outputting at least one image (12) of the captured skin lesion (13) and information (14, 15, 16) associated with it based on the analysis by means of the artificial neural network, wherein the information (14, 15, 16) associated with the image (12) comprises a rendition of an identified predefined class of the skin lesion (14) and/or of a preferably numerical associated risk value (15, 16) of the skin lesion.

The invention relates to a method for generating images to assist in characterizing skin lesions of a human body. In particular, the invention relates to a method for evaluating skin lesions or images of the skin lesions using artificial intelligence.

A method known from the state of the art for identifying skin lesions, i.e., skin changes and skin damage, is dermatoscopy or epiluminescence microscopy, a non-invasive examination technique, in which even deeper skin layers of the areas of skin to be examined can be analyzed using a microscope while illuminating the skin with polarized light. The treating physician makes an assessment or a diagnosis by visually examining the skin lesion in question. The diagnosis can be confirmed by an additional histological examination of the area of skin, which, however, requires surgery to extract a tissue sample.

It is also known for a picture of the skin lesion in question to be taken by means of a video dermatoscope known per se, for example, and to be displayed in an enlarged and/or at least partially changed manner by highlighting specific spectral ranges, for example, in appropriate output means for evaluation by the treating physician. However, the time required for a detailed assessment increases with the number of pictures. In particular if a plurality of captured images of different or similar skin lesions are to be evaluated, as is the case in the course of follow-up examinations, for example, by means of which the changes in a respective skin lesion can be examined and monitored, a reliable and time-efficient characterization of all pictures by the treating physician is no longer possible.

The object of the present invention is to overcome or at least significantly alleviate the disadvantages of the state of the art described above. In particular, an optimized method for identifying and assisting in assessing a skin lesion is to be provided which allows the treating physician to efficiently and reliably identify malignant skin tissue. This object is attained by the subject matter of the independent claims. The dependent claims are advantageous embodiments of the present invention. The invention additionally addresses other issues and proposes solutions for other issues, as is apparent from the following description.

In a first aspect, the invention relates to a method for displaying at least one image of a skin lesion and associated information to assist in characterizing the skin lesion, the method comprising the following steps: capturing a picture, in particular a close-up picture, of a skin lesion in an area of skin to be examined by means of optical capturing means configured for this purpose, in particular a video dermatoscope, and providing image data based thereon, analyzing the skin lesion by electronically processing the provided image data by means of an artificial neural network configured to identify and/or classify skin lesions, and outputting at least one image of the captured skin lesion and information associated with it based on the analysis by means of the artificial neural network, wherein the information associated with the image comprises a rendition of an identified predefined class of the skin lesion and/or a preferably numerical associated risk value or factor of the skin lesion.

The method according to the invention provides a pre-characterization of a skin lesion to be examined which allows the treating physician to efficiently and reliably analyze the skin lesion in question or makes the analysis significantly simpler for the physician. In particular the simultaneous display of an image of the skin lesion in question in combination with information on a class of the skin lesion identified by the artificial neural network and/or an associated risk value assists the treating physician in making an efficient and reliable assessment, in particular if a plurality of skin lesions are to be examined.

The method is preferably at least partially implemented by a software program configured accordingly. The software program can be stored and/or executed on a storage medium or a data carrier, which can be part of an analyzing unit described in more detail below, for example.

The artificial neural network is preferably configured to identify predefined classes or types of skin lesions. In particular, the artificial neural network is configured to identify and/or differentiate at least between non-melanocytic and melanocytic skin lesions. The artificial neural network is preferably configured to identify at least a plurality of the following types of skin lesions as classes:

melanocytic nevus, dermatofibroma, malignant melanoma, actinic keratosis and Bowen's disease, basal-cell carcinoma (basalioma), seborrheic keratosis, solar lentigo, angioma, and/or squamous cell carcinoma.

In a preferred embodiment, the identified classes or types of skin lesions can be output or rendered according to a preferably staged determined probability in addition to and/or based on the analysis of the artificial neural network. For example, the artificial neural network can output not only a most probable class or type of the skin lesion but also the two or three classes or types having the next lower determined probability. In particular, the artificial neural network can output at least two, preferably three or optionally more of the most likely classes or types of the skin lesion to be analyzed. For instance, such an output or rendition can be: basal-cell carcinoma, squamous cell carcinoma, angioma, wherein the individual classes or types are preferably output in the order of decreasing probability. Such an output or rendition permits in particular an efficient analysis of skin lesions which do not appear to be clearly distinguishable from each other and which can be efficiently specified by the information provided.

In a preferred embodiment, the artificial neural network is configured to identify predefined risk classes in particular with respect to a malignity of the skin lesion. A respective risk class can reflect a respective stage of progression of a skin lesion. For example, the artificial neural network can be configured to identify at least two, preferably at least three different stages of progression and therefore risk classes of a respective skin lesion that are associated with them. They can be differentiated as a light, a medium and a severe risk class, for example. A higher risk class can comprise stages of progression of skin lesions that are to be classified as posing a greater risk to the human and which require timely treatment and/or surgery. Furthermore, the artificial neural network is preferably configured to differentiate between a plurality of different stages of progression of a skin lesion type. The classification into corresponding risk classes can be executed by the artificial neural network itself and/or take place by calculation downstream of the processing by the artificial neural network.

In another preferred embodiment, each risk class can also comprise multiple skin lesion types, which can be differentiated or identified by the artificial neural network. A higher risk class can comprise the types of skin lesions that are to be categorized as posing a greater risk to the human. These are in particular classes which require timely treatment and/or surgery. Types posing a lower risk, in particular types of skin lesions that do not require timely treatment and/or surgery, can be categorized as posing a lower risk and therefore be assigned to a low or lower risk class.

The categorization of an identified skin lesion type into a lower or higher risk class can be executed by the artificial neural network and/or take place in another processing or calculation step of the method. For instance, multiple risk classes and skin lesion types comprising them can be stored in a predefined and/or adaptable look-up table. Once a specific skin lesion type and/or a progression of the respective type has/have been identified by the artificial neural network, the associated risk class can be determined and/or calculated and subsequently output.

In a preferred embodiment, a preferably numerical risk value of a given skin lesion is output and/or calculated based on an identified or calculated risk class of skin lesion types and/or based on an identified stage of progression of the skin lesion. The numerical value is preferably between 0.1 and 1. A value between 0.1 and 0.2 can be defined as a low risk value, a value between 0.2 and 0.49 can be defined as a medium risk value, and a value between 0.5 and 1.0 can be defined as a high risk value. The risk value can be calculated by the artificial neural network and/or in another processing or calculation step of the method.

In a particularly preferred embodiment, the outputting or display of at least one image of the captured skin lesion, the analysis of the skin lesion, and/or the display of information associated with the image take(s) place in real time. Furthermore, the image of the captured skin lesion is preferably a live image or a video image of the skin lesion captured or recorded by the capturing means. The video image can be captured or recorded by means of a video dermatoscope, for example, and can be output by means of associated output means, such as a display or a monitor of an analyzing unit, such as a computer, a PC, a tablet or a smartphone, connected to the capturing means. The provision in real time, i.e., without significant delay, permits not only a simplified positioning of the capturing means on the respective skin lesion but also a significantly simplified characterization of the lesion by the treating physician based on the information associated with the shown lesion, which is preferably provided instantly.

The image data provided by the capturing means preferably comprise a plurality of individual images of the skin lesion to be examined. They can be provided by a video dermatoscope as part of a continuous video stream, for example. The provided individual images are preferably each individually analyzed by means of the artificial neural network. The skin lesion to be examined can then be identified and/or classified by the artificial neural network based thereon. The data and information obtained in the process can be used to calculate an overall evaluation result, which will be output as information belonging to the output image of the skin lesion. In particular, a mean value of previously identified individual results or individual classifications of the skin lesion to be analyzed can be formed and subsequently output.

The artificial neural network is preferably what is referred to as a convolutional neural network (CNN), which is known per se. The artificial neural network preferably has at least one hidden layer, more preferably between 1 and 100, most preferably between 1 and 20 hidden layers. In a preferred embodiment, the artificial neural network has between 2 and 10,000, preferably between 2 and 1000 neurons per layer.

The artificial neural network is preferably configured to identify a predefined classification based on knowledge taught by supervised learning. In this process, a large number of skin lesions of different types, different forms and/or different progression according to respective diagnoses are provided to the artificial neural network preferably as image data for teaching in a manner known per se by trained learning. A teaching of this kind can be tested in a manner known per se in a following validation process with respect to the identification precision of the trained artificial neural network. Additionally, an artificial neural network already taught a large database by known transfer teaching can be used and adapted to the respective type of use with few parameter changes. The artificial neural network can be taught and validated using Python Tensorflow and/or Python Keras, for example. Image processing, provision and/or linkage can take place using OpenCV2.4.

The artificial neural network can additionally be configured to further improve previously taught knowledge during the ongoing analysis of the skin lesions from the supplied image data. This means that the artificial neural network is preferably self-learning and continuously adds to and improves its knowledge during the ongoing use in analyzing skin lesions. For instance, information provided by the treating physician on a diagnosis in connection with a captured skin lesion can be taken into account.

In a preferred embodiment, the method comprises the further step of capturing an overview picture of a human body region having a plurality of skin lesions, preferably what is referred to as a clinical picture, and/or the further step of preferably automatically linking a captured close-up picture of a skin lesion with a corresponding skin lesion in a captured overview picture. For example, the overview picture can be a view or a display of a human body part region or a body region, such as a view of the human back. The term close-up picture as used herein refers to a captured image of a single skin lesion, which is preferably captured in close proximity to the skin surface.

The overview picture is preferably captured using the capturing means. In addition to means for taking a respective close-up picture, they can comprise additional means, such as a preferably high-resolution digital photo or video camera for capturing the overview picture. A captured close-up picture can be linked with a respective overview picture manually with the aid of an appropriate input means or automatically by electronic image processing. In particular, this can be made possible by a comparing algorithm which compares the respective pictures with each other and places a corresponding link when it has detected a match of the image data. To do so, feature identification based on an OpenCV library can be used, for example, which is known per se.

In a preferred embodiment, the method comprises the further step of comparing a newly captured picture, in particular a close-up picture, of a skin lesion with previously captured pictures. Based thereon, the captured picture can subsequently be linked with an existing picture as a follow-up picture or newly filed as a first picture of a skin lesion. For the linkage, the respective picture can be compared to existing close-up pictures and to an overview image linked with them. For example, an appropriate algorithm compares a captured close-up picture with existing close-up pictures in the database of the patient in question. If the image evaluation detects matches with existing pictures, this new picture can be marked as a follow-up picture and/or be linked with the lesion in the overview image as a new picture.

In a preferred embodiment, the method comprises the step of analyzing one or more skin lesions by electronically processing a captured overview picture by means of the artificial neural network in order to identify and/or classify the respective skin lesion. In particular, the artificial neural network can be configured to identify and/or classify a plurality of skin lesions in a captured overview picture. The analysis of the skin lesions in an overview picture can take place parallel to or in the background of a respective analysis of a close-up picture of a skin lesion.

In a preferred embodiment, the method comprises the step of outputting information if a close-up picture of a respective skin lesion of a linked overview picture has not yet been captured, in particular if a predefined classification and/or a predefined risk value or risk factor has been determined based on the analysis of the overview picture by the artificial neural network. For example, a warning can be output or a skin lesion in a display of the overview image can be graphically highlighted. Based thereon, the treating physician can capture a close-up picture of the respective skin lesion for closer assessment.

In a preferred embodiment, the method comprises the step of preferably regularly checking a currentness of a respective close-up picture of a skin lesion of a linked overview picture. If a preferably absolute predefined time value of a close-up picture is exceeded for predefined months or years, for example, and/or if currentness values or capturing values of different close-up pictures, such as respective stored capturing dates, differ to a greater extent, information can be output, such as in the form of a warning or graphical highlighting of a skin lesion in a display of the overview image. In doing so, close-up pictures that are older and/or have not been taken anew or updated by the treating physician can be pointed out. This can take place in particular for skin lesions for which a predefined classification and/or a predefined risk factor has been determined based on the analysis of the overview picture by the artificial neural network.

In a preferred embodiment, the artificial neural network is configured to further improve previously taught knowledge during the analysis of the skin lesions in a respective overview picture from the supplied image data. This preferably takes place continuously and can take place parallel to or in the background of an analysis of a respective close-up picture of a skin lesion. For example, information provided by the treating physician on a respective diagnosis in connection with a captured skin lesion can be taken into account.

In a preferred embodiment, the captured pictures of skin lesions are stored in a memory unit, such as an internal or external memory unit of an analyzing unit. They can then be analyzed by means of the artificial neural network in the course of an analysis, which is preferably carried out periodically. In this process, even older pictures can be analyzed with new knowledge of the artificial neural network. In case of deviations of classifications of a skin lesion that are identified in the process from information or diagnoses stored in this regard, a corresponding notification can be output.

The respective images, displays and/or information can be output using output means, such as a display or monitor, of an analyzing unit, such as a computer, a PC, a tablet or a smartphone, connected to the capturing means.

In another aspect, the present invention relates to a diagnosing method for preferably automatically characterizing or assessing skin lesions, the method comprising the following steps:

capturing a picture, in particular a close-up picture, of a skin lesion in an area of skin to be examined by means of optical capturing means configured for this purpose, in particular a video dermatoscope, and providing image data based thereon,

analyzing the skin lesion by electronically processing the provided image data by means of an artificial neural network configured for identifying and/or classifying skin lesions, and outputting at least one image of the captured skin lesion and information associated with it based on the analysis by means of the artificial neural network, wherein the information associated with the image comprises a rendition of an identified predefined class of the skin lesion and/or a preferably numerical associated risk factor of the skin lesion. Thus, a skin lesion to be examined can be automatically diagnosed based on the analysis of an artificial neural network through this method.

The method can additionally comprise other features, which have been described in connection with the method for displaying described above. To avoid redundancies, reference is made to the above description of the method for displaying. In particular, the features described above are also deemed to be disclosed and claimable for the diagnosing method according to the invention and vice-versa.

In another aspect, the present invention relates to a device for implementing a method, in particular a method as described above, the device comprising optical capturing means, in particular a video dermatoscope, for capturing a picture, in particular a close-up picture, of a skin lesion in an area of skin to be examined and providing image data based thereon, an analyzing unit for electronically processing the provided image data by means of an artificial neural network configured to identify and/or classify skin lesions, and output means for outputting at least one image of the captured skin lesion and information associated with it based on the analysis by means of the artificial neural network.

The analyzing unit can be or comprise a computer, such as a PC, a tablet or a smartphone, for example. The analyzing unit preferably comprises at least one internal or external memory unit. The artificial neural network and the data required for operating the network can be stored thereon in a manner known per se. In addition, the analyzing unit is preferably configured to store and execute a software program. The latter can preferably by configured to implement the method according to the invention. The analyzing unit can additionally comprise at least one interface for connecting the capturing means and/or external or additional output means. The analyzing unit can additionally comprise a communication interface for connecting it to an external data server and/or the internet. Furthermore, the analyzing unit can be configured to execute the electronic processing at least partially with the aid and/or based on information provided by external servers and/or database means.

To avoid repetitions, reference is made to the above description of the method according to the invention. In particular, the features of the method described above are also deemed to be disclosed and claimable for the device according to the invention and vice-versa.

Other advantages, features and details of the invention are apparent from the following description of preferred examples of embodiments and from the drawings.

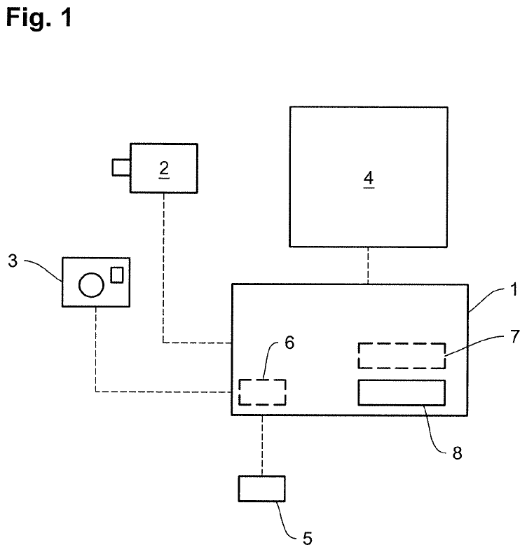

FIG. 1 is a schematic illustration of a preferred embodiment of the device according to the invention;

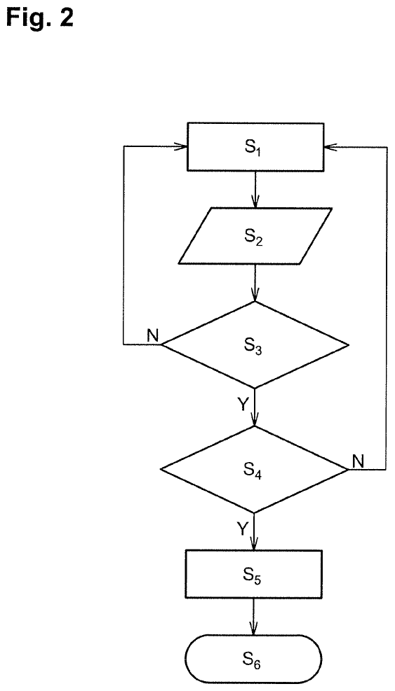

FIG. 2 is a flow diagram of a preferred embodiment of the method according to the invention;

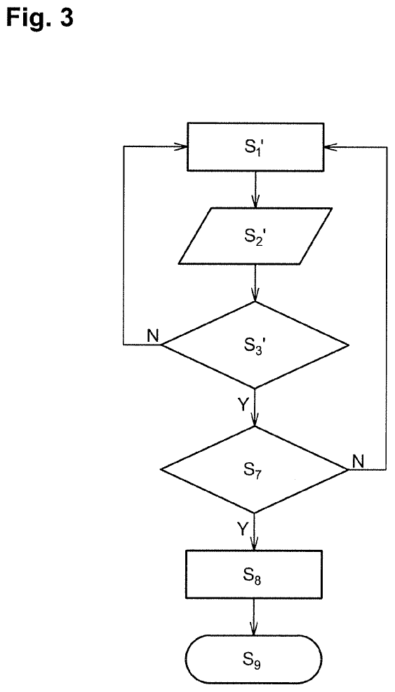

FIG. 3 is a flow diagram of a preferred embodiment of the linkage of a captured close-up picture with a captured overview picture;

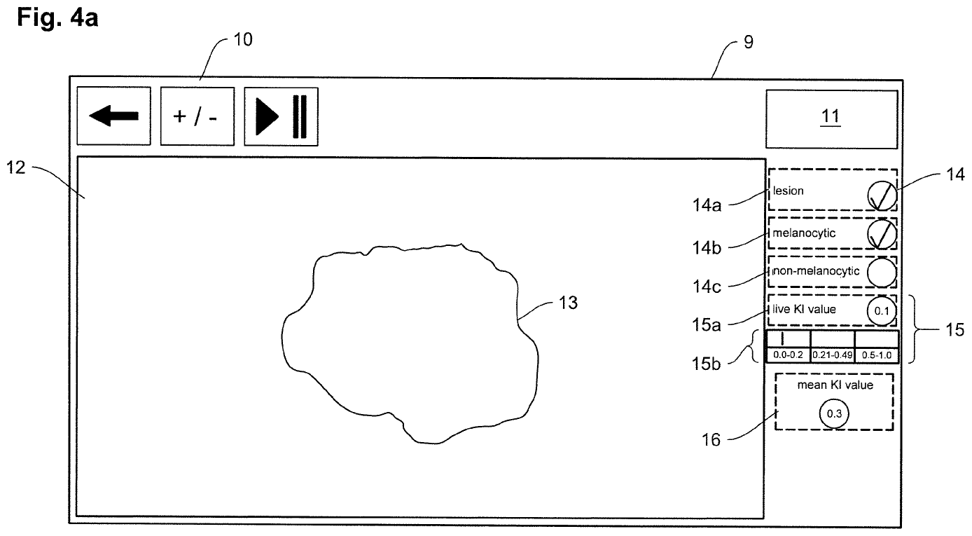

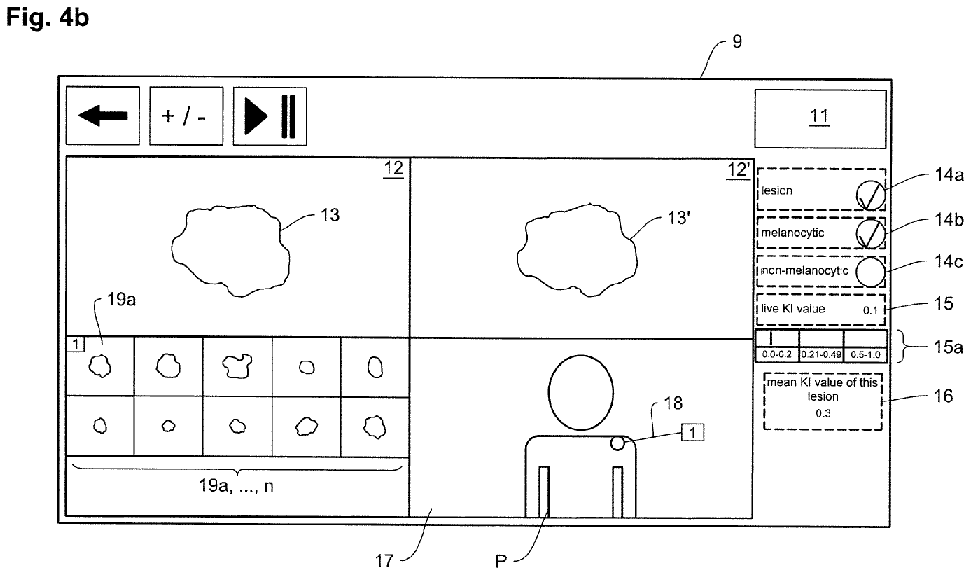

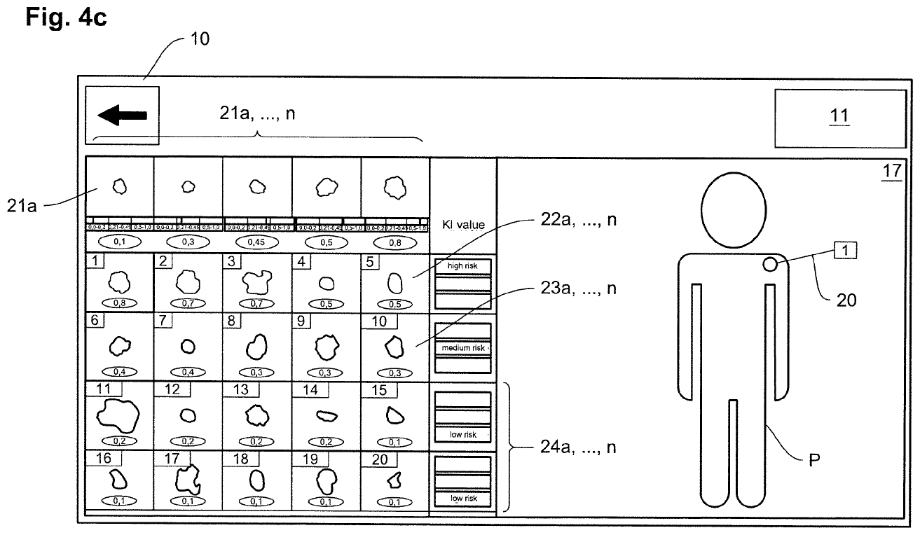

FIG. 4a shows a preferred display in connection with the output of the image of a skin lesion with associated information according to the invention;

FIG. 4b shows a preferred display in connection with the output of the image of a skin lesion and a linkage with an overview picture according to the invention; and

FIG. 4c shows a preferred display in connection with the output of a plurality of images of captured skin lesions and the information associated with them.

FIG. 1 shows a preferred embodiment of a device according to the invention. The device comprises optical capturing means 2, which are configured to capture a picture, in particular a close-up picture, of a skin lesion 13 of a patient P. Capturing means 2 preferably provide digital image data or a signal representing them based on the captured picture. Capturing means 2 preferably comprise a video dermatoscope, which is known per se. The latter can be operated in micro-recording mode to record close-up pictures of a skin lesion. Capturing means 2 can also comprise a preferably high-resolution digital image or video camera 3. The latter can be configured to capture close-up pictures and/or an overview picture of an area of skin of a patient.

The device additionally comprises an analyzing unit 1 for electronically processing the provided image data by means of an artificial neural network. The analyzing unit comprises a processor and/or a memory unit 7. The artificial neural network can be stored and executed thereon for analyzing the image data. The artificial neural network is configured to identify and/or classify skin lesions. To do so, the artificial neural network can access data stored in memory unit 7 and/or access an external server or an external memory unit 5. The latter can be connected to the processor and/or memory unit 7 via a communication interface 6 of analyzing unit 1. Communication interface 6 can additionally be configured to connect capturing means 2 and 3 to analyzing unit 1. Communication interface 6 can enable wireless and/or wired communication with capturing means 2 and 3 and/or the external server or an external memory unit 5.

Analyzing unit 1 is preferably configured to comprise or provide a software suitable in particular for implementing the method according to the invention. The software can be stored and/or executable on the processor and/or memory unit 7. In addition, analyzing unit 1 preferably comprises a user interface for controlling analyzing unit 1 and/or a software executed thereon. The user interface can comprise input means known per se, such as a keyboard, a mouse and/or a touchscreen.

The device additionally comprises output means 4, which are connected to analyzing unit 1 in a wireless or wired manner or comprised by analyzing unit 1. Output means 4 are preferably configured to graphically render information. In particular, output means 4 can comprise a screen and/or a touch display. Output means 4 can additionally be configured to provide acoustic signals or warnings. The output means are in particular configured to provide an image of a captured skin lesion together with at least one associated information based on the analysis by means of the artificial neural network. Analyzing unit 1 is preferably configured to provide the output of the at least one image of the captured skin lesion, the analysis of the skin lesion and/or the display of the information associated with the image in real time.

FIG. 2 shows a flow diagram of a preferred embodiment of the method according to the invention. In a first step (S1), a close-up picture of a skin lesion is captured using capturing means 2. They will subsequently provide an individual image or a video image comprising multiple individual images (S2). In a next step (S3), quality control is performed, in particular as to whether the image quality of the captured picture is sufficient in terms of image resolution and lighting, for example, for being assessed by the artificial neural network. Should the image quality be insufficient because of a lack of resolution of the image, for example, steps S1 to S3 are repeated. If the image quality is sufficient, the associated image data is electronically processed in the course of the analysis by the artificial neural network. The latter can comprise a first step of pre-characterization S4. This step initially determines whether a skin lesion is depicted in the image or not. If it is not a skin lesion, this information can be output and/or steps S1 to S4 can be repeated.

In another step (S5), the artificial neural network precisely identifies and/or classifies the skin lesion. In this process, a specific class or type of the skin lesion can be identified. Also, the artificial neural network can identify a specific progression of the skin lesion. The artificial neural network will provide an output signal corresponding to the identification or classification for further processing. Furthermore, the artificial neural network can be configured to determine or calculate a risk factor based on the identified/classified skin lesion. If the analysis renders the assessment that the skin lesion is suspicious, information to that effect can be output to a user.

Alternatively, the risk factor can be determined or calculated in a subsequent calculation step. This step can make an association with a predefined risk factor or a classification into a predefined value range based on the data provided regarding a type of the skin lesion and/or a progression of the skin lesion. An appropriate software algorithm can be provided for this purpose.

In another step (S6), an image of the captured skin lesion is output together with at least one other information based on the data provided by the artificial neural network. Said information can be at least a pre-characterization parameter or classification information 14 and/or at least a determined risk factor 15, 16.

The method for analyzing the respective picture of the skin lesion preferably takes place in real time. In particular, the method can be implemented by an appropriate software program executed on the analyzing unit described above. The results of the aforementioned steps can be graphically displayed to a user by output means, such as a display and/or output unit.

FIG. 3 shows a flow diagram of a preferred embodiment of how a captured close-up picture is linked with a captured overview picture. One or more overview pictures can be provided by means of capturing means 3 and preferably show a larger skin or body area of a patient, such as a back view or a front view of the upper body. The overview picture can also be a back view or a front view of the entire body. The overview picture preferably shows a plurality of skin lesions and allows captured close-up pictures to be linked to make them easier to find again when follow-up pictures are taken, for example.

In shown steps S1′ to S3′, close-up pictures of a skin lesion are captured, a corresponding image or video image is provided and quality is subsequently controlled, in particular in terms of image resolution and lighting, analogously to steps S1 to S3 described above. In a subsequent step (S7), the provided image data of the close-up picture is compared to the image data of at least one previously captured overview image. An algorithm for this purpose, which is known per se, compares said image data and automatically links the individual image with the overview image if a match is found. If no match is found, the image can be stored as a new image and/or another close-up picture (St to S3′) can be captured. The appropriate linkage will take place in another step (S8). The linkage can be output directly or displayed to a user as a suggestion for explicit confirmation. The result of the linkage can be displayed in another step (S9). For example, the close-up picture can be arranged at an appropriate position in the overview picture and/or a mark can be placed in the overview image and linked with the close-up picture. A user will be able to click such a position in an overview picture to open the linked close-up picture, for example.

FIGS. 4a to 4c show a preferred display by means of output means 4 of a device according to the invention, i.e., screenshots of a graphical interface 9 of a software program configured to implement the method according to the invention.

The output, i.e., graphical interface 9, comprises an image 12 of the captured skin lesion 13. Preferably, a live image, i.e., a real-time stream of a video recording, which is being captured using a video dermatoscope, for example, is displayed. The output, i.e., graphical interface 9, preferably comprises navigation and/or control means 10, by means of which different views and/or zoom levels of image 12 can be selected, for example. Also, the output, i.e., graphical interface 9, preferably comprises information on the patient 11 to be examined. The output, i.e., graphical interface 9, additionally comprises information 14, 15, 16 which is associated with a skin lesion 13 depicted and which is based on the identification and/or classification by the artificial neural network. At least some of said information 14, 15, 16 is preferably provided in real time.

The output information can in particular comprise (pre-)classification information. The latter can comprise an indicator or a displayed parameter 14a which indicates whether the analyzed skin anomaly or the captured area of skin is or includes a skin lesion 13. Furthermore, the classification information can comprise an indicator or a displayed parameter 14b, 14c which indicates whether the analyzed skin lesion is melanocytic or non-melanocytic. Furthermore, the output information can comprise an identified class or type of the skin lesion to be analyzed in each case. For example, the output can indicate whether the skin lesion is a melanoma, a nevus, a basal-cell carcinoma, etc.

The information can also comprise two or three of the classes or types of the skin lesion to be analyzed that have been identified as the most probable by the artificial neural network, preferably in the descending order of probability.

The output information can additionally comprise a rendition of a risk factor 15, which indicates the health relevancy or the risk posed by the analyzed skin lesion. Risk factor 15 can be output in numerical and/or graphical form 15a. Alternatively or additionally, a predefined comparison scale 15b can be graphically rendered. The latter can at least be divided into low, medium and high risk. The aforementioned information is preferably output in real time based on the captured skin lesion and the analysis by the artificial neural network.

The output Information can additionally comprise a mean value and/or an average risk factor in numerical and/or graphical form 16. Said risk factor can be based on multiple individual analyses of a captured skin lesion if multiple individual images of a specific skin lesion are captured and analyzed, for example.

FIG. 4b shows how a newly captured close-up picture 12 is linked with a previously captured overview picture 17. As described above with reference to FIG. 3, close-up picture 12 is automatically linked with an overview picture 17 of patient P through an image comparison. The result of the linkage can be displayed in a separate window or section of the graphical interface. In particular, a match in a plurality of lesions 19a, . . . , 19n identified in an overview picture 17 can be highlighted (19a). Additionally, an indicator 18 in overview picture 17 can indicate the corresponding position on the body and in the overview picture. The result of the automatic linkage can preferably be manually adjusted by the user if the automatic linkage is not correct, for example.

Next to a preferably live display of captured skin lesion 13 in image 12, a reference close-up picture 12′, i.e., the last close-up picture 12′ captured for the lesion in question in the overview image, can be displayed. Newly captured close-up picture 12 can be identified as a follow-up picture and stored accordingly. In this way, image history data for a respective lesion or a respective position in an overview picture can be recorded.

FIG. 4c shows a preferred display when a plurality of images of captured skin lesions and information associated with them are output. A lesion history consisting of a plurality of close-up pictures 21a, . . . , 21n which have been captured at different points in time, such as during respective examinations, can be displayed for each of the captured skin lesions. Additionally, associated information, such as a risk factor, can be displayed and/or a link with an overview picture 17 can be provided by means of a position indicator 20 for each of the close-up pictures. The display can additionally comprise a division of the respective lesion into risk classes, such as high, medium or low. Lesions for which a high risk factor has been determined (22a, . . . , 2n) can be displayed separately or highlighted for a user. The other risk classes, such as medium (23a, . . . , 23n) and low (24a, . . . , 24n) can also be displayed.

This displaying method allows a treating physician to check the lesions posing a particular risk more closely at regular intervals. Additionally, the artificial neural network can be configured to analyze the respective skin lesions in the existing image database of a patient parallel to or in the background of a respective examination of close-up pictures. This can in particular also take place based on the captured overview pictures. In this process, changes in lesions in the overview picture can be identified and/or checked. Additionally, it can be checked whether the user will continue to examine a respective lesion by epiluminescence microscopy, i.e., by capturing close-up pictures. If this is not the case, the method or the device can be configured to alert the user to possibly suspicious lesions and/or to suggest returning to examining a suspicious lesion by epiluminescence microscopy.

The embodiments described above are of a purely exemplary nature, and the invention is by no means limited to the embodiments shown in the figures.

### REFERENCE SIGNS

1 analyzing unit
2 capturing means
3 capturing means for overview picture
4 output means
5 external server/data memory
6 communication interface
7 processor/memory unit
8 user interface
9 graphical interface
10 navigation/control means
11 patient information
12, 12′ image of skin lesion (close-up picture)
13, 13′ skin lesion
14a-c (pre-)classification information
15a numerical rendition of risk value
15b graphical rendition of risk value
16 average risk value
17 captured overview image (overview picture)
18 indicator for automatic link
19a display of linked skin lesion
19b, . . . , n display of alternatively linkable skin lesions
20 position indicator
21a, . . . , n lesion history
22a, . . . , n high risk lesion overview
23a, . . . , n medium risk lesion overview
24a, . . . , n low risk lesion overview
P patient/area of skin
S1, S1′ capturing picture
S2, S2′ image/video image
S3, S3′ quality control
S4 pre-characterization step
S5 identification and/or classification
S6 assessment output
S7 image comparison
S8 image linkage
S9 outputting linkage result

1. A method for displaying at least one image of a skin lesion and associated information to support the characterization of the skin lesion, comprising the steps: capturing a picture of a skin lesion (13) in an area of skin to be examined by optical capturing means (2) and providing image data based thereon,
analyzing the captured picture of the skin lesion by electronically processing of the provided image data provided by an artificial neural network configured to identify or classify the skin lesion, and
outputting at least one image (12) of the skin lesion (13) represented in the captured picture and information (14, 15, 16) associated with it based on the analysis by the artificial neural network,
wherein the information (14, 15, 16) associated with the at least one image (12) comprises a rendition of an identified predefined class of the skin lesion (13) or a numerical associated risk value (15, 16) of the skin lesion (13).

2. The method according to claim 1, wherein the artificial neural network is configured to identify predefined classes of skin lesions, or the classes melanocytic nevus, dermatofibroma, malignant melanoma, actinic keratosis and Bowen's disease, basal-cell carcinoma (basalioma), seborrheic keratosis, solar lentigo, angioma, or squamous cell carcinoma.

3. The method according to claim 1, wherein the artificial neural network is configured to identify predefined risk classes with respect to a malignity of the skin lesion (13), or wherein the analysis of the skin lesion (13) comprises calculating a risk value (15, 16) based on an identified risk class of the skin lesion.

4. The method according to claim 1, wherein the outputting of the at least one image (12) of the captured skin lesion (13), the analysis of the skin lesion, or the displaying of the information (14, 15, 16) associated with the image takes place in real time.

5. The method according to claim 1, wherein the image data comprises at least two individual images of the skin lesion (13), each of which is analyzed by the artificial neural network, and wherein an overall evaluation result (16) of the individual images is calculated in order to output the information associated with the images.

6. The method according to claim 1, wherein the artificial neural network is configured to identify a predefined classification based on knowledge taught by supervised learning, or wherein the artificial neural network is configured to further improve previously taught knowledge while analyzing the skin lesion from the supplied image data.

7. The method according to claim 1, wherein the artificial neural network is a convolutional neural network (CNN), or wherein the artificial neural network has at least one hidden layer.

8. The method according to claim 1, wherein the method further comprises the following steps:
capturing an overview picture (17) of a human body region comprising a plurality of skin lesions, or automatically linking a close-up picture (12) of a skin lesion with a corresponding skin lesion in a captured overview picture (17).

9. The method according to claim 8, wherein the method further comprises the step of comparing a newly captured picture of a skin lesion (12) with previously captured pictures (12′), and the picture is linked as a follow-up picture or newly filed as a first picture of a skin lesion based thereon.

10. The method according to claim 8, wherein the method comprises the step of analyzing one or more skin lesions (13) by electronically processing the captured overview picture (17) by means of the artificial neural network in order to identify or classify the respective skin lesion.

11. The method according to claim 10, wherein the method comprises displaying information if a close-up picture has not been captured yet of a respective skin lesion for which a predefined classification or a predefined risk value has been determined based on the analysis of the overview picture by the artificial neural network.

12. The method according to claim 11, wherein the method comprises checking a currentness of a respective close-up picture belonging to an overview picture (17) and outputting information if a predefined time value has been exceeded and/or in the event of deviations from currentness values of close-up pictures (12, 12′).

13. The method according to claim 8, wherein the artificial neural network is configured to further improve previously taught knowledge during the analysis of the skin lesions in the overview picture (17) from the supplied image data.

14. The method according to claim 1, wherein the captured pictures are stored in a memory unit (7), and the stored pictures are periodically analyzed by means of the artificial neural network.

15. The method according to claim 1, wherein the respective image and/or the respective information is output by output means.

16. A diagnostic method for characterizing skin lesions according to claim 1, wherein an identified predefined class of the skin lesion (13) or a numerical associated risk value (15, 16) is output to characterize the skin lesion (13).

17. A device for implementing a method according to claim 1, comprising: optical capturing means (2) for capturing a picture of a skin lesion (13) in an area of skin to be examined and providing image data based thereon,
an analyzing unit (1) for electronically processing the provided image data by an artificial neural network configured to identify or classify skin lesions, and output means (4) for outputting at least one image (12) of the skin lesion (13) captured in the picture and information (14, 15, 16) associated with it based on the analysis by the artificial neural network.
